SEQUENCE LISTING (4 sequences)

SEQ ID NO: 1 - RNA, 19 nt, synthetic construct
  source 1..19: mol_type = other RNA
ggctaccttgttacgactt

SEQ ID NO: 2 - RNA, 18 nt, synthetic construct
  source 1..18: mol_type = other RNA
cggtgaatacgttcccgg

SEQ ID NO: 3 - RNA, 20 nt, Turicimonas muris
  source 1..20: mol_type = unassigned RNA
gcggttttgcaagatggatg

SEQ ID NO: 4 - RNA, 23 nt, Turicimonas muris
  source 1..23: mol_type = unassigned RNA
gcactctagtcttgcagtcatgt

(19) United States
(12) Patent Application Publication
(10) Pub. No.: US 2026/0207681 A1
(43) Pub. Date: Jul. 23, 2026

(54) COMPOSITION AND USE OF TURICIMONAS MURIS FOR THE TREATMENT OF METABOLIC DISEASES

(71) Applicants: SORBONNE UNIVERSITE, PARIS (FR); INSTITUT NATIONAL DE LA SANTE ET DE LA RECHERCHE MEDICALE, PARIS (FR); ASSISTANCE PUBLIQUE - HOPITAUX DE PARIS, PARIS 12 (FR)

(72) Inventors: Karine CLEMENT, PARIS (FR); Tiphaine LE ROY, PARIS (FR); Jean DEBEDAT, PARIS (FR); Lise VOLAND, PARIS (FR)

(21) Appl. No.: 19/142,278

(22) PCT Filed: Dec. 21, 2023
(86) PCT No.: PCT/EP2023/087217

§ 371 Date: Jun. 23, 2025

Related U.S. Application Data
(63) Continuation of application No. PCT/FR2022/052482, filed on Dec. 22, 2022.

(51) Int. Cl. A61K 35/741; A61K 9/00; A61K 31/155; A61K 31/365; A61K 31/4015; A61K 31/485; A61K 31/702; A61K 31/7048; A61K 38/26; A61K 45/06; A61P 3/04; A61P 3/10

(52) U.S. Cl. CPC A61K 35/741; A61K 9/0053; A61K 31/155; A61K 31/365; A61K 31/4015; A61K 31/485; A61K 31/702; A61K 31/7048; A61K 38/26; A61K 45/06; A61P 3/04; A61P 3/10

(57) ABSTRACT

The present disclosure relates to the prevention and/or treatment of metabolic diseases such as overweight and obesity as well as complications related thereto. The present disclosure more specifically relates to the bacterium Turicimonas muris or fragments thereof for preventing and/or treating metabolic diseases and complications related thereto.

The present invention relates to the prevention and/or treatment of metabolic diseases, such as, for example, overweight and obesity, as well as complications related thereto. More specifically, the present invention relates to the bacterium Turicimonas muris or fragments thereof for preventing and/or treating metabolic diseases and complications related thereto.

The obesity is defined as an excessive accumulation of fat mass or adipose tissue (AT) that can impair health and lead to the development of numerous pathologies (type 2 diabetes: T2D, cardiovascular diseases, dyslipidemia, cancer, etc.). This excessive fat gain results from the interplay of genetic, epigenetic, dietary, and environmental factors that contribute to the imbalance of energy balance.

The clinical diagnosis of obesity is traditionally based on the calculation of the body mass index (BMI, weight in kilograms divided by height in meters squared). In adults, a BMI comprised between 18.5 and 24.99 kg/m² is considered normal. The overweight is defined by a BMI comprised between 25 and 29.99, while a BMI ≥30 defines the obesity. The severity of obesity is classified into three grades: grades I (moderate obesity; 30≤BMI≤35), II (severe obesity; 35≤BMI≤40), and III (morbid obesity; BMI ≥40). According to the World Health Organization, 39% of adults worldwide are overweight, and 13% are obese.

Several studies report a strong, positive relationship between a high BMI and an increased risk of developing a T2D. In this sense, the individuals suffering from obesity are up to 80 times more likely to develop a T2D compared to those with a BMI below 25 kg/m². The T2D is characterized by a chronic hyperglycemia due to an insulin resistance in peripheral tissues (liver, skeletal muscle, adipose tissue) combined with insufficient insulin secretion by pancreatic β-cells. Its clinical diagnosis is based on the demonstration of fasting blood glucose ≥1.26 g/L on two occasions or ≥2 g/L at any time of the day. The T2D significantly impairs quality and length of life, particularly through its vascular complications such as microangiopathies (nephropathy, retinopathy and neuropathy) and macroangiopathies (stroke and myocardial infarction). According to Sanjay Basu et al., nearly 406 million people worldwide suffered from T2D in 2018 (BASU et al. The Lancet. Diabetes & Endocrinology. January 2019, Vol. 7, No. 1, pp. 25-33).

These chronic diseases worsen over time and significantly reduce life expectancy. They therefore constitute a major public health problem with a significant economic and societal burden. The medical cost of obesity represents 1 to 3% of healthcare expenditures in most countries (OECD. Health Goals. October 2010. https://www.oecd.org/fr/els/systemes-sante/46044602.pdf).

For patients with obesity and associated metabolic pathologies, a pharmacological approach can be proposed in addition to dietary rebalancing and the promotion of regular physical activity. These drug approaches are non-curative and aim to control symptoms, slow the progression of the disease, and limit the risk of complications. To date, five obesity treatments—orlistat, phentermine-topiramate, naltrexone-bupropion, liraglutide, and semaglutide—have been approved by the Food and Drug Administration in the United States for long-term use (Basu et al. The Lancet. Diabetes & Endocrinology. January 2019, Vol. 7, No. 1, pp. 25-33). In France, only orlistat and, more recently, liraglutide have received approval for use in the treatment of obesity.

The T2D treatments, on the other hand, primarily aim to improve insulin sensitivity (metformin) or potentiate insulin secretion by pancreatic b cells (sulfonylureas, glinide, semaglutide). More recently, gliflozins, or SGLT2 inhibitors, which cause increased glycosuria, may be proposed.

An insulin therapy should be initiated when the lifestyle and dietary approaches and the non-insulin treatments fail to achieve the glycemic target (glycated hemoglobin level ≤7%) (HAS. Drug strategy for glycemic control in Type 2 diabetes, January 2013. https://www.hassante.fr/uploa/docs/application/pdf/2023-02/10irp04_synth_diabete_type_2_objectif_glycemique_messages_cles.pdf).

The current lifestyle, dietary, and drug-based approaches fail to effectively and sustainably treat most patients with obesity and associated diseases. New therapeutic targets and treatments must therefore be developed to improve patient care, slow the progression of these diseases, and prevent the development of complications.

Over the past two decades, a growing number of studies have identified the gut microbiota (GM) as a new player in the pathophysiology of obesity and associated metabolic disorders. The microbiota is an ecosystem consisting of a set of microorganisms (viruses, bacteria, archaea, fungi, and protozoa) living in a given ecological niche, in this case, the digestive system. The recent studies estimate that the adult digestive tract (DT) is home to approximately 100 trillion bacteria, belonging to approximately 4,600 species (Almeida et al. Nature Biotechnology. January 2021, Vol. 39, No. 1, pp. 105-114).

The GM bacteria perform essential biological functions: they participate in the metabolism of xenobiotics and indigestible foods, synthesize vitamins, produce certain bioactive metabolites (for example the short-chain fatty acids—SCFAs), and are essential for the maturation and the activation of the immune system. A myriad of studies in humans and mice report a strong and positive correlation between high bacterial richness (or alpha diversity) in the GM, a metabolic homeostasis, and the absence of inflammation. Conversely, the obesity and the T2D are pathologies associated with a reduction in microbial diversity and dysbiosis of the GM, that is to say a persistent imbalance in the composition and functions of the bacterial community.

With the emergence of culturomics, that is to say high-throughput culture to characterize the microbial composition of the GM, and with the recent optimization of bacterial culture media, a growing number of strains have been isolated, cultured in pure conditions, and characterized. These advances pave the way for in vitro studies of the metabolic potential of these bacteria and in vivo studies of supplementation with bacteria of interest. They thus make it possible to study the causal relationships between certain bacterial species of the GM and the modulation of the host's physiological state and to identify the involved cellular and molecular mechanisms.

To date, a limited number of isolated species and strains (less than ten) have been characterized and identified as microorganisms with a direct causal effect on the metabolic health. The mechanisms involved, even partial, have only been identified for an even smaller number of bacteria. These results, which are based on studies made possible only by pure bacterial culture, demonstrate the undeniable causal role of certain GM bacteria in regulating the host's energy metabolism. The isolation and functional analysis of new bacterial strains potentially beneficial to the host are therefore necessary in order to consider effective and non-invasive therapeutic strategies aimed at treating, or even preventing, obesity and associated metabolic pathologies.

In this context, the Inventors have demonstrated the beneficial effect of the bacterium Turicimonas muris on the energy metabolism and the physiology in mice; more particularly, the administration of Turicimonas muris reduces the weight gain and fat mass induced by an obesogenic diet. The beneficial effects of this bacterium on the body composition of mice on a high-fat diet (HFHS) are associated with improved glucose tolerance compared to control mice, fed under the same conditions. It appears that the administration of Turicimonas muris has a beneficial effect on the body composition and carbohydrate metabolism of mice without affecting their food intake or the amount of excreted stools. The inventors have also demonstrated a correlation between the relative abundance of Turicimonas muris measured in stools in human subjects and the good metabolic state of these subjects (in particular a decrease in blood sugar).

The present invention thus relates to Turicimonas muris for its use in the prevention and/or treatment of metabolic diseases and/or the complications related thereto.

It also relates to the use of Turicimonas muris to improve glucose and fat metabolism, increase energy expenditure, and/or promote weight loss, particularly in overweight or obese individuals.

The species Turicimonas muris (Taxonomy ID: 1796652) was isolated from the cecal contents of mice by Lagkouvardos et al. during the establishment of the «Mouse Intestinal Bacterial Collection», a public repository consisting of 100 bacterial strains isolated from the cecal contents of mice with conventional microbiota (SPF status for Specific Pathogen Free) (LAGKOUVARDOS et al. Nature Microbiology. October 2016, Vol. 1, No. 10, p. 16131). This Gram-negative bacterium grows optimally at temperatures ranging from 20 to 45° C. (mesophilic) and is also present in the human GM. It belongs to the Sutterellaceae family and the Proteobacteria phylum (henceforth Pseudomonadota).

The present invention also relates to bacterial strains belonging to the same family as Turicimonas muris or to genetically modified Turicimonas muris strains that retain the same properties as Turicimonas muris.

The Turicimonas muris bacterium can be cultured as described in the experimental section.

For the purposes of the present invention, it can be used in viable or non-viable form, as well as in pasteurized or freeze-dried form. It can also be used in fragment form. The term «fragment» refers to cellular components, metabolites, secreted molecules, and compounds resulting from the metabolism of Turicimonas muris. The fragments may be obtained, for example, by recovering the supernatant of a Turicimonas muris culture or by extracting cellular components or cell fractions, metabolites, or secreted compounds from a Turicimonas muris culture. The term «fragment» may also refer to a degradation product, in particular metabolites.

According to one embodiment, Turicimonas muris or its fragments are substantially purified. As used herein, the term «substantially purified» means that Turicimonas muris or its fragments are comprised in a sample where they represent at least about 50%, preferably at least about 60, 70, 80, 85, 90, 95, 99%, or more of the bacterial strains or their fragments by weight of said sample.

The term «metabolic disease» refers to disorders, diseases, and conditions caused by or characterized by weight gain, abnormal energy utilization or consumption, altered responses to ingested or endogenous nutrients, energy sources, hormones, or other signaling molecules in the body, or altered metabolism of carbohydrates, lipids, proteins, nucleic acids, or a combination thereof. A metabolic disease may be associated with a deficiency or excess in a metabolic pathway resulting in an imbalance in the metabolism of carbohydrates, lipids, proteins, and/or nucleic acids.

The examples of metabolic diseases comprise, but are not limited to, metabolic syndrome, disorders of insulin deficiency or resistance, diabetes mellitus (such as, for example, type 2 diabetes), impaired glucose tolerance, abnormal lipid metabolism (or dyslipidemia), overweight, and obesity; complications of metabolic diseases (also referred to as comorbidities) comprise in particular the atherosclerosis, hypertension, preeclampsia, cardiac disease, stroke, non-alcoholic fatty liver disease, hyperglycemia, fatty liver disease of various etiologies, immune system dysfunction associated with overweight and obesity, cardiovascular disease, hypercholesterolemia, elevated triglycerides, asthma, sleep apnea, osteoarthritis, neurodegeneration, gallbladder disease, syndrome X, inflammatory and immune disorders, atherogenic dyslipidemia, and cancer, in particular, esophageal cancer, gastric cardia cancer (a type of stomach cancer), bowel cancer, liver cancer, gallbladder cancer, pancreas, breast cancer in postmenopausal women, breast cancer, ovarian cancer, kidney cancer, meningioma (brain tumor), thyroid cancer, and multiple myeloma.

The amounts of Turicimonas muris to be administered are preferably selected to restore a normal bacterial population in the intestine, that is to say a population such as that found in a healthy subject (not overweight or obese).

For example, the administered amount of Turicimonas muris may be selected between approximately 1·10² and 1·10¹⁵ cfu (colony forming unit), in particular between approximately 1·10⁴ and 1·10¹² cfu or approximately 1·10⁶ and 1·10¹⁰ cfu; this amount is preferably administered daily. When Turicimonas muris is administered in a non-viable form or as fragments, the aforementioned amounts are used for the preparation of this form. The administered amount of Turicimonas muris may also be in the range of approximately 1·10⁶ to approximately 1·10¹² cells, preferably between approximately 1·10⁸ and approximately 1·10¹⁰ cells, or between approximately 1·10⁹ and approximately 1·10¹⁰ cells; this amount is preferably administered daily.

The present invention also relates to a composition comprising Turicimonas muris and a physiologically acceptable carrier.

The term «physiologically acceptable carrier» means a carrier that can be administered and well tolerated by an individual, particularly a mammal, preferably a human.

It may be mentioned for examples, without being exhaustive, bulking agents such as corn starch; and carrier agents such as potato starch. The composition may be incorporated into a capsule of plant origin; the composition may also comprise anti-caking agents such as magnesium stearate or silicon dioxide.

The composition according to the invention comprises Turicimonas muris in viable, non-viable or fragment form, in an amount comprised between approximately 1·10² and 1·10¹⁵ cfu, in particular between approximately 1·10⁴ and 1·10¹² cfu or between approximately 1·10⁶ and approximately 1·10¹⁰ cfu or between approximately 1·10⁶ and approximately 1·10¹² cells, preferably between approximately 1·10⁸ and approximately 1·10¹⁰ cells or between approximately 1·10⁹ and approximately 1·10¹⁰ cells.

Turicimonas muris or a fragment thereof or the composition according to the invention can be administered by several routes of administration.

Examples of suitable routes of administration comprise, but are not limited to, oral administration, rectal administration, administration by esophagogastroduodenoscopy, administration by colonoscopy, administration using a nasogastric or orogastric tube, etc. preferably, the administration is by oral or rectal route.

According to one embodiment, Turicimonas muris or a fragment thereof or the composition of the invention is in a form suitable for oral administration. According to this embodiment, the form suitable for oral administration may be:
a solid form selected from the group consisting of tablets, pills, capsules, soft gelatin capsules, sugar-coated pills, orodispersible tablets, effervescent tablets, or other solids; according to a particular embodiment, the oral solid form is gastro-resistant so that its contents are not degraded during its stay in the stomach.
a liquid form, such as, for example, an oral solution, liposomal forms, and the like.

When administered orally, the composition of the invention may be a nutritional composition or a food product.

According to one embodiment, Turicimonas muris or a fragment thereof or the composition of the invention is in a form suitable for rectal administration. According to this embodiment, the composition may be a suppository or a rectal capsule.

According to one embodiment, the composition of the invention further comprises a probiotic and/or a prebiotic. According to this embodiment, the composition of the invention is preferably administered orally.

The term «probiotic» refers to microbial cell preparations (such as, for example, live microbial cells) which, when administered in an effective amount, have a beneficial effect on the health or well-being of a subject. By definition, all probiotics are proven non-pathogenic. In one embodiment, these health benefits are associated with improving the balance of human or animal microbiota in the gastrointestinal tract, and/or restoring normal microbiota.

For example, the probiotic can be selected from:
the bacteria: Lactobacillus, Lactococcus, Bifidobacterium, Veillonella, Desemzia, Christensenella, Allobaculum, Coprococcus, Collinsella, Citrobacter, Turicibacter, Sutterella, Subdoligranulum, Streptococcus, Sporobacter, Sporacetigenium, Ruminococcus, Roseburia, Proteus, Propionobacterium, Leuconostoc, Weissella, Pediococcus, Streptococcus, Prevotella, Parabacteroides, Papillibacter, Oscillospira, Melissococcus, Dorea, Dialister, Clostridium, Cedecea, Catenibacterium, Butyrivibrio, Buttiauxella, Bulleidia, Bilophila, Bacteroides, Anaerovorax, Anaerostopes, Anaerofilum, Enterobacteriaceae, Fermicutes, Atopobium, Alistipes, Acinetobacter, Slackie, Shigella, Shewanella, Serratia, Mahella, Lachnospira, Klebsiella, Idiomarina, Fusobacterium, Faecalibacterium, Eubacterium, Enterococcus, Enterobacter, Eggerthella;

In particular, Bifidobacterium animalis, in particular Bifidobacterium animalis spp. lactis, Bifidobacterium lactis, Lactobacillus rhamnosus, Lactobacillus casei, Lactobacillus acidophilus, Akkermansia muciniphila;
the prokaryotic microorganisms: Archaea, Firmicutes, Verrucomicrobia, Christensenella, Bacteroidetes (such as, for example, Allistipes, Bacteroides ovatus, Bacteroides splachnicus, Parabacteroides, Prevotella Bacteroides stercoris, ruminicola, Porphyromondaceae), Proteobacteria, Betaproteobacteria (such as, for example, Aguabacterium and Burkholderia), Gammaproteobacteria (such as, for example, Xanthomonadaceae), Actinobacteria (such as, for example, Actinomycetaceae and Atopobium), Fusobacteria, Methanobacteria, Spirochaetes, Fibrobacteres, Deferribacteres, Deinococcus, Thermus, Cyanobacteria, Methanobrevibacteria, Peptostreptococcus, Ruminococcus, Coprococcus, Subdoligranulum, Dorea, Bulleidia, Anaerofustis, Gemella, Roseburia, Dialister, Anaerotruncus, Staphylococcus, Micrococcus, Propionobacteria, Enterobacteriaceae, Faecalibacterium, Bacteroides, Parabacteroides, Prevotella, Eubacterium, Bacilli (such as for example, Lactobacillus salivarius, Aerococcus, Granulicatella, Streptococcus bovis and Streptococcus intermedius), Clostridium (such as for example, Eubacterium hallii, Eubacterium limosum) and Butyrivibrio.
the yeasts: Ascomycetes, Zygomycetes and Deuteromycetes, in particular groups Aspergillus, Torulopsis, Zygosaccharomyces, Hansenula, Candida, Saccharomyces, Clavispora, Bretanomyces, Pichia, Amylomyces, Zygosaccharomyces, Endomycess, Hyphopichia, Zygosaccharomyces, Kluyveromyces, Mucor, Rhizopus, Yarrowia, Endomyces, Debaryomyces, and Penicillium.

In particular, the following strains can be cited:
Lactobacillus type: L. acidophilus, L. amylovorus, L. casei, L. gasseri, L. helveticus, L. johnsonii, L. pentosus, L. plantarum, L. reuteri and L. rhamnosus
Bifidobacterium type: B. adolescentis, B. animalis, B. bifidum, B. breve, B. infantis and B. longum.
Other lactic acid bacteria: Enterococcus faecium, Lactococcus lactis and Streptococcus thermophilus;
Other microorganisms: Bacillus clausii, Escherichia coli Nissle 1917, and Saccharomyces cerevisiae (boulardi).

Preferably, the probiotic (or LBP) is selected from: Escherichia coli K12, Christensenella minuta, Anaerobutyricum soehngenii, Dysosmobacter welbionis, and Akkermansia muciniphila, Faecalibacterium prausnitzii, Afnia alvei 4597, and P. goldsteinii.

For the purposes of the present invention, the probiotic may be used in viable or non-viable form, or in fragment form. It is administered in the same amounts as those indicated for Turicimonas muris.

The term «prebiotic» refers to a substance, such as, for example, a substance that cannot be digested by humans but that modulates the composition and/or activity of the intestinal microbiota through its metabolism by intestinal microorganisms, thus conferring a beneficial physiological effect on the host.

The prebiotics can be dietary fiber. The dietary fiber can be selected from the group consisting of the fructo-oligosaccharides (FOS), the galacto-oligosaccharides (GOS), the xylo-oligosaccharides, the isomalto-saccharides, the soy oligosaccharides, the pyrodextrins, the transgalactosylated oligosaccharides, the lactulose, the beta-glucan, the inulin, the raffinose, and the stachyose. The dietary fiber also has the advantage of being resistant to several conditions, in particular heating and long storage periods. They may further contribute to treatment within the scope of the present invention by improving gastrointestinal health and increasing satiety.

Other non-limiting examples of prebiotics comprise the water-soluble cellulose derivatives, the water-insoluble cellulose derivatives, the unprocessed oat flakes, the psyllium, the wheat bran, and mixtures thereof. Examples of water-soluble cellulose derivatives comprise in particular, the methylcellulose, the methylethylcellulose, also the hydroxyethylcellulose, the ethylhydroxyethylcellulose, the cationic hydroxyethylcellulose, as well as the hydroxypropylcellulose, the hydroxyethylmethylcellulose, but also the hydroxypropylmethylcellulose and the carboxymethylcellulose.

The composition according to the invention may be administered in combination with a drug intended for the treatment of obesity, such as orlistat, phentermine-topiramate, naltrexone-bupropion, liraglutide, semaglutide and tirzepatide and/or in combination with a drug intended for the treatment of type 2 diabetes such as metformin, sodium-glucose co-transporter type 2 inhibitors, gliptins, acarbose, glinides, sulfonamides or even sulfonylureas such as the Glimeperide.

The present invention also relates to the (non-therapeutic) use of Turicimonas muris to promote the weight loss in an individual as well as to a method for preventing and/or treating metabolic diseases and their complications comprising the administration of Turicimonas muris or its fragments or a composition according to the invention.

### FIGURES

FIG. 1—T. muris is predominantly present in the ileum of non-obese mice.

qPCRs were performed with specific (T. muris) and nonspecific (total bacteria) primers on jejunal (Figures a to c) and ileal (Figures d to f) content samples from mice fed a control diet (chow diet) or a HFD supplemented or not with FOS and/or 5-ASA.
(a, d) Bacterial load for each group of mice.
(b, e) T. muris concentration.
(c, f) Relative abundance of T. muris.
Data were analyzed using the Kruskal Wallis test followed by Dunn's pairwise multiple comparisons test and are represented as scatter plots with the mean±SEM. Number of mice per group: n=5 to 12. *q<0.05; **q<0.01; ***q<0.001; ****q<0.0001.

FIG. 2—T. muris prevents diet-induced obesity.

Diagram of the experimental device. The animals in two of the four groups were fed a HFHS diet, and those in the remaining two groups were fed a control diet (chow diet). The mice in one of the HFHS and chow diet groups were supplemented once daily with T. muris, with its freeze-dried culture supernatant, or with culture medium uninoculated with T. muris.
(b, c) Body weight and body weight gain.
(d, e) Fat mass and fat mass gain. Weight (in mg) of different fat depots after the sacrifice:
(f) SAT: subcutaneous (inguinal) AT,
(g) EAT: epididymal AT,
(h) BAT: brown AT.

The data in Figures b and d were analyzed using a two-way ANOVA followed by Tukey's post hoc test. Data in Figures c, e, f, g, and h were analyzed using the Kruskal-Wallis test followed by Dunn's pairwise multiple comparisons test. The results are presented as histograms with the mean±SEM. Number of mice per group: n=9. *q<0.05; **q<0.01; ***q<0.001; ****q<0.0001 for HFHS vs. HFHS-T. muris comparisons and “q<0.01; 000 q<0.001; 000 q<0.0001 for chow diet vs HFHS comparisons.

FIG. 3—T. muris prevents diet-induced alterations in carbohydrate metabolism.

Fasting blood glucose measured during week 3.

Glycemic profile and

Area under the curve measured during an oral glucose tolerance test (OGTT) performed during week 10.

The results in figures a and c were analyzed using the Kruskal Wallis test followed by Dunn's post hoc test. The results in figure b were analyzed using a 2-way ANOVA followed by Tukey's post hoc test. Number of mice per group: n=9. *q<0.05; **q<0.01; ***q<0.001 for HFHS vs. HFHS-T. muris comparisons and” q<0.01; 000 q<0.0001 for chow diet vs HFHS comparisons.

FIG. 4: The relative abundance of Turicimonas muris was measured by quantitative PCR in the stools of individuals with either normal (18<BMI<25) or obese (BMI>30) body mass index, as well as in individuals initially suffering from obesity and type 2 diabetes before undergoing bariatric surgery (Roux-en-Y bypass) and who had, or had not, entered remission of their type 2 diabetes on average 5 years after the surgery.
(A) Relative abundance of T. muris (% of total bacteria) as a function of the body mass index. Mann-Whitney test.
(B) Proportion of individuals with detectable levels of T. muris by quantitative PCR (that is to say greater than 0.00009% of total bacteria) as a function of the body mass index. Chi-square test.
(C) Relative abundance of T. muris as a function of fasting blood glucose in obese patients (BMI>30). The threshold for moderate fasting hyperglycemia (1.1 g/L) is indicated. Overall, T. muris is not detected in patients with moderate fasting hyperglycemia, while it is detected in a proportion of patients with normal fasting blood glucose levels.
(D) Relative abundance of T. muris in patients initially suffering from obesity and type 2 diabetes who have entered, or have not, remission of their type 2 diabetes. T. muris is not detected in patients still suffering from diabetes, while it is detected in a proportion of patients who have entered remission. Mann-Whitney test.

### EXAMPLE

### I. Materials and Methods

Bacterial Culture of Turicimonas muris:

Culture medium: The powders (Table 1) were dissolved in distilled water, then the formic acid, rezasurin solution (a colored redox indicator), and hemin stock solution were added. The pH was adjusted to 7.8. The resulting medium was transferred into glass vials and boiled in a microwave in order to remove the dissolved oxygen. The vials were capped and autoclaved. At the same time, the temperature-sensitive ingredients (Table 2) were transferred into empty vials. The vials were capped, a mixture of CO₂, H₂, and N₂ was injected using a needle, then a second needle was inserted into the septum to evacuate the O₂ present in the air. Anaerobic water, FCS, and the vitamin stock solution were added to the powders. Three ml of the resulting solution and a filter-sterilized menadione solution (80 ng/ml final) were added to each of the medium vials. The latter were finally pressurized by injecting the filter-sterilized CO₂, H₂, and N₂ mixture and stored at 4° C.

TABLE 1
culture medium composition

| Ingredients per 11 culture mediums | Amount | |
|---|---|---|
| Yeast Extract | 5 | g |
| Soy peptone | 10 | g |
| NaCI | 2.5 | g |
| Na₂CO₃ | 1.5 | g |
| Formic Acid 80% | 1.5 | ml |
| Resazurin Solution (1 g/L) | 1 | ml |
| KH₂PO₄ | 1.33 | g |
| Na₂CO₃ | 1.5 | g |
| NH₄Cl | 1.5 | g |
| Hemin Solution (20 mg/ml) | 66.5 | μl |
| H₂O | 900 | ml |

TABLE 2
culture medium composition (ingredients added sterilely
by filtration after thermal sterilization of the medium)

| Autoclave-sensitive | | |
|---|---|---|
| ingredients per 1 L of medium | Amount | |
| Galactose | 3 | g |
| Ascorbic acid | 0.5 | g |
| Reduced glutathione | 1 | g |
| Vitamin Mix Solution | 0.2 | ml |
| Cysteine | 1 | g |
| Na₂CO₃ | 1.2 | g |
| Anaerobic water | 60 | ml |
| FCS | 20 | ml |

Inoculation and administration: Turicimonas muris (DSM 22575) was purchased from the German Public Collection of Microorganisms and Cell Cultures (DSMZ) and cultured under sterile, anaerobic conditions for 48 hours at 37° C. For the administration to mice, the bacterial culture was centrifuged at 4000 g for 20 min at 4° C. The bacterial pellets were resuspended in 5-fold concentrated culture supernatant by freeze-drying. These preparations, measured by qPCR at 5·109 cfu (colony-forming units)/ml, were finally stored at −80° C. in glycerol until force-feeding of the mice.

Animal models: 36 8-week-old male C57BL/6J SPF mice (Charles River Laboratories, France) were housed in a controlled environment (temperature 22±2° C., 12-h light/dark cycle) with free access to food and water. The mice were acclimated for 7 days while being fed a control diet (chow diet, 3.91 kcal/g, 4% fat, Research Diet, ref. 98121701) and without being handled. After this acclimatization, the mice were identified by ear tag and 4 groups of 9 mice of similar weight and body composition were formed. Mice in two of the four groups were fed the control diet for 12 weeks, while those in both groups were fed a diet enriched with lipids and sucrose (high-fat,high-sucrose diet: HFHS, 4.68 kcal/g, 21% fat and 50% carbohydrates, Research diet, ref. D12079B).

In order to demonstrate the impact of Turicimonas muris on host physiology, the bacterium was administered by oral force-feeding once daily in the form of live cells resuspended in culture supernatant (200 μL of the solution containing Turicimonas muris, or 1·10⁹ cfu). The control mice received the uninoculated culture medium, also concentrated 5-fold by freeze-drying. The force-feeding of the animals began 1 week after their arrival at the animal facility, at the same time as the diet change for the HFHS groups.

### Mice Sacrifice:

An intracardiac blood sample was taken under isofluorane (3%) anesthesia immediately prior to euthanasia by cervical dislocation of the 36 mice. The tissues and organs of interest were collected, weighed, and frozen in liquid nitrogen (liver, spleen, inguinal, epididymal and interscapular AT, ileum and ileal contents, jejunum and jejunal contents, colon, gastrocnemius muscle). Before embedding the various tissues/organs in paraffin using the HISTOMICS ICM—Pitié Salpêtrière platform, the liver and jejunum were fixed in paraformaldehyde (4%) for histological analyses.

All experiments were approved and conducted in accordance with the guidelines of the Charles Darwin ethics committee.

### Body Composition Analysis:

In order to monitor the development of obesity, the body composition of the mice (fat mass, lean mass, and fluids) was determined by nuclear magnetic resonance (NMR-LF90 Minispec+scanner) at the time of feeding, then every 3 weeks, and the day before sacrifice.

### Oral Glucose Tolerance Test (OGTT):

The 6-hour fasted mice received an oral glucose load (2 g of glucose per kg of body weight). The blood glucose levels were measured using a glucometer (Accu Check ROCHE) from a drop of blood collected with a 23G needle from the lateral tail at times −30, 0.15, 30, 60, and 90 min post-carbohydrate loading. At times 0 and 15 min, 30 μL of blood was collected from the tail using an EDTA-coated capillary tube to perform an insulin assay.

### Transit Time Measurement:

The mice received 200 μL of carmine red solution (concentration 10 mg/ml) by oral force-feeding. The transit time corresponds to the period elapsed between force-feeding and the appearance of the first red stool.

### Food Intake:

The mice's food intake was determined throughout the experiment by calculating the difference in weight between the food placed in the cage and the food remaining after one week. To determine daily feed intake per cage and per mouse, the following formula is used:

$$\frac{\begin{matrix}Weight of feed placed in the cage - \\ Weight remaining after one week\end{matrix}}{\begin{matrix}Number of days between the 2 measurements / \\ number of animals per cage\end{matrix}}$$

The results are expressed in grams of feed/day/animal and in kilocalories/day/animal.

### Excreted Calories:

The excreted calories will be determined by fecal calorimetry. To do this, the mice were placed for 3 days during week 11 in cages equipped with a mesh bottom allowing to collect stools. All of the mice's feces were collected over 3 days and then placed in a 60° C. incubator to dehydrate them. The collected feces were then weighed, and the amount of energy contained per gram of stools was measured using a bomb calorimeter. The percentage of absorbed calories, that is to say the proportion of ingested energy that was not excreted in the feces, is calculated using the following formula:

$$\% of absorbed calories = 100 * \frac{Calories in 1 g of feces * fecal mass}{Calories in 1 g of food * ingested food mass}$$

Calories in 1 g of food * ingested food mass. The feed efficiency is determined by calculating the ratio: weight gain/absorbed calories.

DNA Extraction from Mouse Feces and Digestive Contents—Adapted GODON Protocol:

### Bacterial Lysis:

The samples of jejunal and ileal contents were weighed and placed on ice. 250 μL of 4M guanidine thiocyanate, 350 μL of 6% sarcosine, and 30 μL of DTT were added to each tube. Once vortexed, the contents were transferred to Precellys-compatible tubes and incubated for 15 minutes at 95° C. To enhance cell lysis, silica beads with diameters of 0.1 and 2 mm were added to the samples. The tubes were then shaken six times for 30 seconds, interspersed with 30 seconds of rest, at a frequency of 10,000 Hz, and then left on ice for 2 minutes.

### Nucleic Acid Isolation and Protein Removal

20 mg of PVPP (polyvinylpolypyrrolidone) were then homogenized and 600 μL of TEN-PVPP buffer (a solution containing PVPP, Tris HCl, EDTA, and NaCl) were added to each tube. The samples were centrifuged, and the supernatant was collected. The steps: TEN-PVPP, centrifugation, and supernatant collection were repeated 3 times. Under a fume hood, a (1:1) ratio of phenol, chloroform, and isoamyl alcohol (25:24:1) was added to the supernatants. The tubes were vortexed and centrifuged; the aqueous phase was collected. The samples were then incubated for 1 hour at 60° C. with 15 μL of proteinase K (19 mg/mL). After centrifugation, the nucleic acids present in the supernatant were precipitated with isopropanol and left on ice for 20 minutes. The tubes were then mixed by inversion, centrifuged, and the supernatant was removed. The pellets, previously air-dried, were gently resuspended in phosphate buffer and potassium acetate. After a centrifugation, the supernatants were collected.

### RNA Digestion and Purification

The samples were incubated for 45 min at 37° C. with RNase. The DNA was precipitated with absolute ethanol and sodium acetate. The samples were then centrifuged 3 times. Between each centrifugation, the supernatant was removed and the pellet was washed with 500 μL of 70% ethanol. The pellets were finally air-dried and resuspended in 60 μL of TE.

The 260/230 nm and 260/280 nm OD ratios (indicators of nucleic acid purity) as well as the DNA concentration were determined using a NanoDrop1000 (Thermo Fisher Scientific, USA). The samples were finally stored at −20° C.

### Human Stool DNA Extraction

The fecal DNA was extracted from stool using a PureLink™ Microbiome DNA Purification Kit (Invitrogen, Paris, France) according to the protocol provided by the manufacturer.

Quantification of T. Muris by Quantitative Polymerase Chain Reaction (qPCR):

The extracted bacterial DNA was diluted to a concentration of 10 ng/μL. 2.5 μL of diluted DNA and 7.5 μL of master mix were added to the bottom of a 96-well ABI FAST plate.

Master mix composition for one reaction: Fast Sybr Green Applied Biosystems 4385612 (5 μL), forward and reverse primers (200 nM), BSA (2.5 μg), and water (qsp 10 μL).

The plates were then centrifuged (1 min at 800 rpm), placed in the qPCR machine (StepOnePlus System), and the program execution (StepOne v2.3 software) was launched. To determine the absolute bacterial concentration, a standard range was applied to each PCR plate by diluting the genomic DNA of a pure Turicimonas muris culture of known concentration. This range varied from 4·10⁵ to 26 cfu per well. The relative Turicimonas muris concentration was determined by dividing the absolute Turicimonas muris concentration by the absolute total bacterial concentration.

Thermal cycler program: first, one 5-minute cycle at 95° C. for initial denaturation, followed by a second 30-second denaturation step at 95° C., an annealing step at 60° C. for 30 seconds, and an elongation step at 72° C. for 30 seconds. All carried out

| | | | |
|---|---|---|---|
| Non-specific primers | Sense | 5′ GGC-TAC-CTT-GTT-ACG-ACT-T 3′ | SEQ ID No. 1 |
| (quantification of | Antisens | 5′ CGG-TGA-ATA-CGT-TCC-CGG 3′ | SEQ ID No. 2 |
| total bacteria) | | | |
| Turicimonas muris | Sense | 5′ GCG-GTT-TTG-CAA-GAT-GGA-TG 3′ | SEQ ID No. 3 |
| primers | Antisens | 5′ GCA-CTC-TAG-TCT-TGC-AGT-CAT-GT 3′ | SEQ ID No. 4 |

### Statistical Analyses

The statistical analyses were performed using GraphPad Prism software version 7.00 for Windows (GraphPad Software, San Diego, CA, USA). The data distribution was verified using the Shapiro-Wilk test. The comparisons between groups at a given time point were performed using a one-way ANOVA followed by a Tukey post hoc test for parametric data or a Kruskal-Wallis test followed by Dunn's correction for nonparametric data. The comparisons between the groups at different time points were performed using a two-way ANOVA followed by the Tukey post hoc test. For all graphs, data are represented as the mean±SEM (standard error mean). A corrected q-value≤0.05 was considered statistically significant.

### II. Results

II.1. Study of the Intestinal Localization of Turicimonas Muris in Mice with a Complex Microbiota

To study the localization of Turicimonas muris throughout the intestine, its abundance was quantified by qPCR using ileal and jejunal content samples from mice with complex microbiota. For this experiment, the mice were divided into five groups:

The chow diet group, corresponding to the control group in which the mice were fed a control diet for 15 weeks (same duration for all groups).

The HFD group, in which the mice were fed a fat-enriched diet to induce obesity (60% of calories from fat).

Finally, the mice in the last three groups, HFD/FOS, HFD/5-ASA (5-aminosalicylic acid), and HFD/FOS/5-ASA, were fed a HFD diet supplemented with FOS and/or 5-ASA in the drinking water.

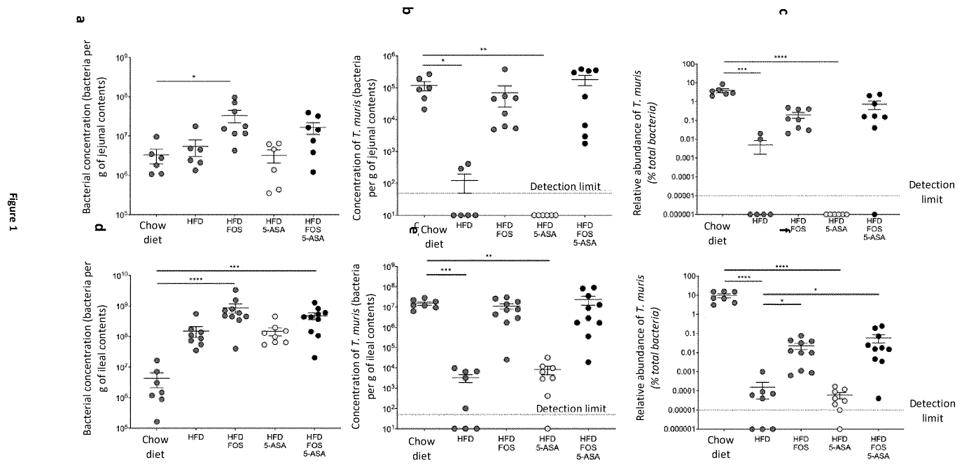

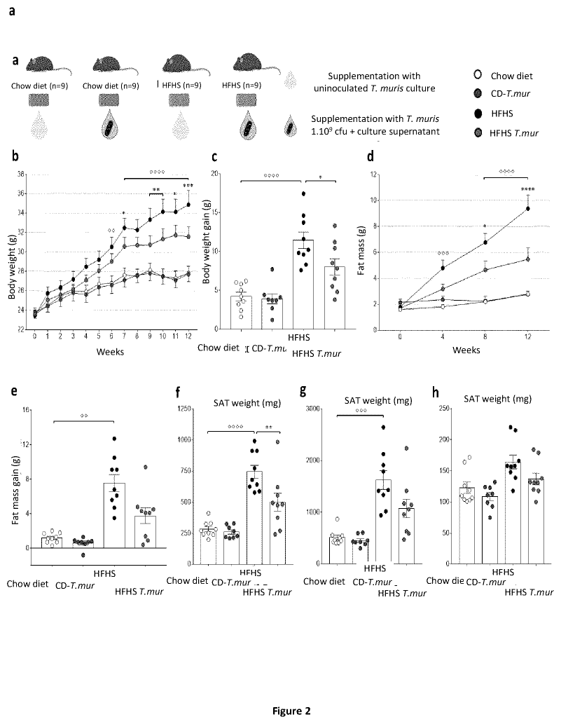

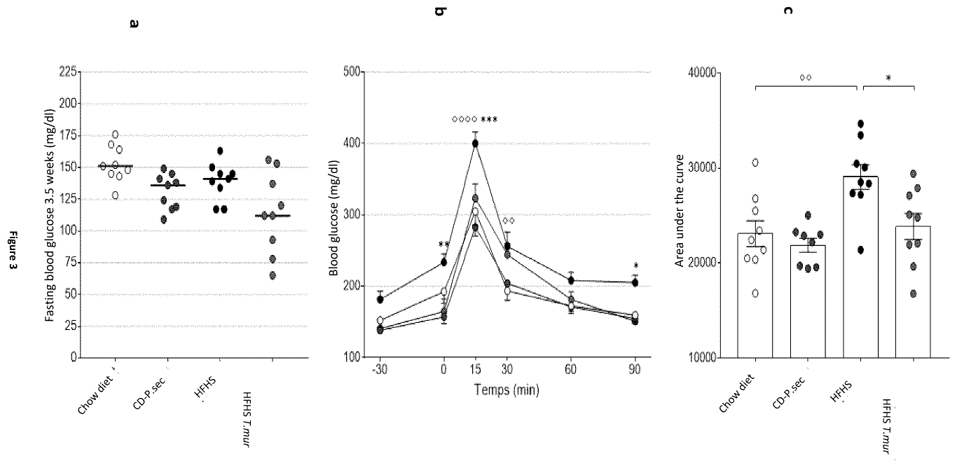

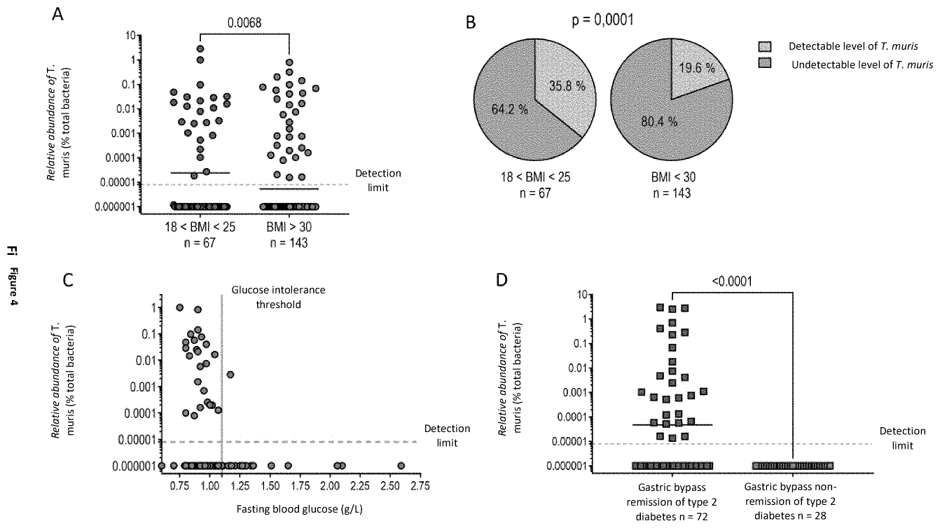

As expected, FOS treatment increased the bacterial load in the intestinal contents of mice fed the HFD diet (treated or not with 5-ASA) compared to that of untreated mice fed under the same conditions (FIGS. 1a and d).

In both ileal and jejunal content samples, Turicimonas muris was predominantly detected in the chow diet, HFD/FOS, and HFD/FOS/5-ASA groups (FIGS. 1 b and e). Similar trends were observed in the relative abundance of T. muris (FIGS. 1 c and f). Thus, unlike 5-ASA, which has no influence on the proliferation of Turicimonas muris in the intestine, FOS is a prebiotic that partially restores its abundance in the intestinal contents of mice with diet-induced obesity (DIO).

The relative abundance of Turicimonas muris was higher in the ileal contents (7.6%) than in the jejunal contents (4.3%) of mice fed a chow diet (FIGS. 1 c and f). These results confirm those published by Andrew J. Macpherson et al. showing that Turicimonas muris is predominantly present in the ileum of mice with a simplified Oligo-MM12 microbiota.

### II.2. The Administration of Turicimonas Muris Prevents Obesity Induced by the Obesogenic Diet

In order to induce obesity and impaired carbohydrate metabolism, mice in two of the four groups (18 out of 36 mice) were fed a high-fat, high-sucrose (HFHS) diet for 12 weeks. Mice in the remaining two groups received a control diet (chow diet).

In order to demonstrate the impact of Turicimonas muris on host physiology, regardless of the involved mechanisms, it was administered by oral force-feeding to half of the mice in each group in the form of cells (for cellular components) resuspended in culture supernatant (for secreted metabolites). The control mice received the uninoculated culture medium, also concentrated by freeze-drying (FIG. 2 a).

From 6 weeks of follow-up, data show that mice fed a HFHS diet gained significantly more weight than those fed a control diet. This difference increased throughout the experiment (FIG. 2 b). NMR analyses indicate that the increase in body weight in these HFHS mice was primarily due to an increase in fat mass rather than lean mass (FIGS. 2 d and e). Overall, these results confirm the efficacy of the DIO model.

Interestingly, the Turicimonas muris administration limited the body weight and fat mass gain induced by the HFHS diet (FIGS. 2 b and d). Compared with untreated HFHS mice, the body weight and fat mass gain of HFHS-Turicimonas muris mice was significantly lower, by 3.4 and 3.8 g, respectively (FIGS. 2 c and e). The difference between the groups in terms of body weight gain and fat mass remained substantially unchanged when the mice were on the control diet, showing that the effect of Turicimonas muris is observable only in the context of obesity induced by the fat- and sucrose-enriched diet. It should be noted that the lean mass of the mice was not affected by either the diet or Turicimonas muris administration (data not shown).

The observations made on the weight and body composition of the mice were corroborated by the tissue weights measured during animal dissection. Indeed, the mass of subcutaneous AT (SAT) and epididymal AT (EAT) fat deposits in mice fed the HFHS diet was significantly greater than that of mice fed the control diet (FIGS. 2 f and g). Although not significant, similar trends were observed for BAT (FIG. 2 h). The administration of Turicimonas muris to the mice limited the increase in the weight of these different adipose depots. Again, the positive effect of this bacteria was limited to the TAs of mice under HFHS.

Finally, neither Turicimonas muris administration nor the diet had any effect on the mass of the gastrocnemius muscle, liver, and spleen of the mice (data not shown). These results demonstrate that Turicimonas muris administration counteracts the development of obesity induced by the obesogenic diet by limiting body weight and fat mass gain in the mice.

### II.3. The Turicimonas Muris Administration Prevents the Alteration of Carbohydrate Metabolism Induced by the Obesogenic Diet

The lower adiposity of mice fed HFHS and treated with Turicimonas muris was associated with reduced fasting blood glucose (FIG. 3a) and improved glucose tolerance, as shown by the OGTT performed during week 10 and the resulting area under the curve (FIGS. 3b and c). The HFHS-T. muris mice indeed had lower hyperglycemia induced by the glucose load (time 15 min to 90 min) compared to untreated HFHS mice (FIG. 3b).

### II.4. Impact of Turicimonas Muris Administration on Energy Balance and Digestive Physiology

The weight and fat mass gain occur when the calories supplied by the diet and absorbed by the intestines exceed those excreted and expended by the body.

It was then assessed whether the beneficial effect of Turicimonas muris administration on body composition and carbohydrate metabolism was the result of a decrease in food intake and/or nutrient absorption in the intestine and/or an increase in energy expenditure.

It was found that the average daily food intake (in g) of mice on the chow diet was significantly higher than that of mice fed the HFHS diet (FIG. 2 a). Given that the caloric content is higher for the HFHS diet, the resulting amount of energy absorbed was identical for all groups (FIG. 2 b). This food intake, as well as the transit time of the mice, was not significantly impacted by Turicimonas muris administration (FIGS. 2 a, b, and c).

Finally, the daily stool weight per mouse varied depending on the diet but was not affected by the administration of the bacterium. It was indeed greater in mice on the chow diet than in those fed the obesogenic diet (FIG. 3 d).

Overall, these data indicate that Turicimonas muris administration beneficially impacts the body composition and carbohydrate metabolism of mice without affecting their food intake or stool excretion. These latter observations suggest that Turicimonas muris was well tolerated by the mice.

### III. Conclusions

The obtained results demonstrate a beneficial effect of Turicimonas muris on the energy metabolism and the physiology of mice.

More specifically, the Turicimonas muris administration reduces the weight and fat mass gain induced by the obesogenic diet. The beneficial effects of this bacterium on the body composition of mice on HFHS are associated with improved glucose tolerance compared to control mice, fed under the same conditions. Finally, it appears that the administration of Turicimonas muris has a beneficial effect on the body composition and carbohydrate metabolism of mice without affecting their food intake or the amount of excreted stool.

### Relative Abundance of Turicimonas Muris was Measured by Quantitative PCR in the Stool of Human Subjects.

The relative abundance of Turicimonas muris was measured by quantitative PCR (see «Materials and Methods» section above) in the stool of individuals with either a normal body mass index (18<BMI<25) or an obese body mass index (BMI>30), as well as in individuals initially suffering from obesity and type 2 diabetes before undergoing bariatric surgery (Roux-en-Y bypass) and who had, or had not, entered remission of their type 2 diabetes on average 5 years after surgery.

The obtained results are presented in FIG. 4.

In humans, the detection of T. muris in stool is associated with improved metabolic status in several contexts.

First, the mean relative abundance of T. muris is significantly higher in stools from patients with normal body mass index than in stools from patients with obesity (FIG. 4A). This is also translated into a higher prevalence of T. muris in the microbiota of patients with normal body mass index since T. muris is detected by quantitative PCR in 35.8% of these patients while it is only detected in 19.6% of patients with obesity (FIG. 4B). The prevalence of T. muris is also associated with better carbohydrate metabolism since, in a population of patients with obesity, it is detected almost exclusively in patients with fasting blood glucose levels below 1.1 g/L (FIG. 4C). Finally, in patients initially suffering from obesity and type 2 diabetes, T. muris was detected only in a portion of patients who had entered remission of their type 2 diabetes following bariatric surgery. It was not detected in any patient still suffering from type 2 diabetes after bariatric surgery (FIG. 4D). This negative association of the abundance and presence of T. muris with the corpulence and blood glucose levels of the patients demonstrates a positive effect of T. muris on the metabolism of the patients.

1. Turicimonas muris for use in the prevention and/or treatment of metabolic diseases and/or the complications related thereto.

2. Turicimonas muris for use according to claim 1, wherein it is in viable, non-viable, or fragment form.

3. Turicimonas muris for use according to claim 1, wherein it is administered in an amount comprised between 1·10² and 1·10¹⁵ cfu.

4. A composition comprising Turicimonas muris and a physiologically acceptable carrier.

5. The composition according to claim 4, wherein it comprises between 1·10² and 1·10¹⁵ cfu of Turicimonas muris.

6. The composition according to claim 4, wherein it further comprises at least one probiotic and/or at least one prebiotic.

7. The composition according to claim 6, wherein it comprises at least one probiotic selected from Escherichia coli K12, Christensenella minuta, Anaerobutyricum soehngenii, Dysosmobacter welbionis, and Akkermansia muciniphila, Faecalibacterium prausnitzii, Afnia alvei 4597, and P. goldsteinii.

8. The composition according to claim 4, wherein it is administered orally or rectally.

9. The composition according to claim 4, for use in the prevention and/or treatment of metabolic diseases and/or the complications related thereto.

10. The composition according to claim 4, for use in the prevention and/or treatment of metabolic diseases and/or the complications related thereto, wherein it is administered in combination with a medicament intended for the treatment of obesity, such as orlistat, phentermine-topiramate, naltrexone-bupropion, liraglutide, semaglutide, and tirzepatide.

11. The composition according to claim 4, wherein it is administered orally or rectally, and wherein it is administered in combination with a medicament intended for the treatment of type 2 diabetes, such as metformin, sodium-glucose cotransporter type 2 inhibitors, gliptins, acarbose, glinides, sulfonamides, or sulfonylureas such as Glimeperide.

12. A use of Turicimonas muris to promote weight loss in an individual.

13. Turicimonas muris for use according to claim 2, wherein it is administered in an amount comprised between 1·10² and 1·10¹⁵ cfu.

14. The composition according to claim 5, wherein it further comprises at least one probiotic and/or at least one prebiotic.

15. The composition according to claim 5, wherein it is administered orally or rectally.

16. The composition according to claim 6, wherein it is administered orally or rectally.

17. The composition according to claim 7, wherein it is administered orally or rectally.
